SEQUENCE LISTING (2 sequences)

SEQ ID NO: 1 - DNA, 23 bp, Artificial Sequence
  Description of Artificial Sequence Synthetic primer
gccaggtgcctccacagtcagcc

SEQ ID NO: 2 - DNA, 29 bp, Artificial Sequence
  Description of Artificial Sequence Synthetic primer
tgtgtgtgcccatccgctctgagctccac

(19) United States
(12) Patent Application Publication
(10) Pub. No.: US 2009/0270484 A1
(43) Pub. Date: Oct. 29, 2009

(54) WWOX Vectors and Uses in Treatment of Cancer

(75) Inventors: Carlo M. Croce, Columbus, OH (US); Muller Fabbri, Columbus, OH (US); Francesco Trapasso, Columbus, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: The Ohio State University Research Foundation, Columbus, OH (US)

(21) Appl. No.: 12/083,067

(22) PCT Filed: Oct. 4, 2006
(86) PCT No.: PCT/US06/38824

§ 371 Date: Jun. 20, 2008

Related U.S. Application Data
(60) Provisional application No. 60/723,752, filed on Oct. 5, 2005.

(51) Int. Cl. A61K 48/00; C12N 15/87; C12N 15/11; C12N 15/00; C12N 5/06

(52) U.S. Cl. 514/44 R; 435/455; 536/23.5; 435/320.1; 435/325

(57) ABSTRACT

The present invention provides novel methods and compositions for the diagnosis, prognosis and treatment of cancer in a subject, by administering to the subject a polynucleotide encoding a functional WWOX gene product.

### GOVERNMENT SUPPORT

This invention was supported, in whole or in part, by grants from NCI/NIH Grant/Contract Number CA78890, CA77738 and CA56036. The Government has certain rights in this invention.

### FIELD OF INVENTION

The invention generally relates to compositions and methods for controlling abnormal cell growth, including but not limited to, that found in cancer, and in particular, lung cancer.

### BACKGROUND OF THE INVENTION

Lung cancer is the leading cause of cancer mortality in the United States (1), with an incidence of about 170,000 new cases per year in the United States (1), and mortality is very high. Nonsmall cell lung cancer (NSCLC) accounts for about 80% of lung cancers. Surgery remains the main therapy for NSCLC, but a large fraction of patients cannot undergo curative resection. Despite new drugs and therapeutic regimens, the prognosis for lung cancer patients has not significantly changed in the last 10 years. Recombinant virus gene therapy has been investigated in lung cancer patients; adenovirus (Ad) and retrovirus encoding wild-type p53 have been injected intratumorally in lung cancer clinical trials (2-6). Recombinant Ad injection in lung cancer phase I studies (7) has demonstrated safety and feasibility, and phase I/II clinical trials are currently recruiting patients to evaluate toxicity and efficacy of gene therapy with recombinant Ads.

Lung cancer is associated with early loss of expression of the FHIT (fragile histidine triad) gene (8) at fragile site FRA3B (9). Fragile regions are particularly susceptible to damage on exposure to environmental carcinogens, which are etiological factors in lung cancer. Recently, Yendamuri et al. (10) have demonstrated that the WWOX (WW domain containing oxidoreductase) gene is also altered in a fraction of nonsmall cell lung cancers. WWOX is located at fragile site FRA16D (11) and encodes a 414-aa protein with two WW domains and a short-chain dehydrogenase domain. WW domains are protein-protein interaction domains, and Wwox interactors with important signaling roles in normal epithelial cells have been identified. Wwox interacts with p73 and can trigger redistribution of nuclear p73 to the cytoplasm, suppressing its transcriptional activity (12). Wwox also interacts with Ap2-γ transcription factors with roles in cell proliferation (13). Most recently, Wwox has been reported to compete with Yap protein for binding to the intracellular ErbB4 domain, a transcriptional activator (14). Thus, the Wwox pathway includes a number of downstream signaling proteins that may also serve as cancer therapeutic targets.

The WWOX gene is altered in many types of cancer, including breast, ovary, prostate, bladder, esophagus, and pancreas (15-19). In nonsmall cell lung cancer, transcripts missing WWOX exons were detected in 26% of tumors and in five of eight cell lines (10). WWOX allele loss occurred in 37% of tumors, and the promoter is hypermethylated in 62.5% of squamous cell lung carcinomas (10, 19). To investigate tumor suppression in lung cancer, we studied in vitro and in vivo effects of Wwox protein expression in Wwox-negative (A549, H460, and H1299) and -positive lung cancer cells (U2020) by infection with Ad carrying the WWOX gene; H1299 cells were also stably transfected with an inducible Wwox expression vector, which allows induction of near physiologic levels of protein. Wwox restoration effectively induced apoptosis in vitro and suppressed lung cancer tumorigenicity in nude mice, with no effect on lung cancer cells that constitutively express the Wwox protein.

### SUMMARY OF THE INVENTION

The invention provides methods for treating cancer in a subject, comprising administering to the subject a polynucleotide encoding a functional WWOX gene product. In some embodiments, the cancer is chosen from lung cancer, breast cancer, ovarian cancer, prostate cancer, bladder cancer, esophageal cancer, and pancreatic cancer. In some embodiments, the administration comprises gene therapy, and in some embodiments, recombinant viral gene therapy, such as recombinant adenoviral gene therapy.

The invention further provides methods of treating cancer in a subject comprising inducing Wwox expression in at least one cancer cell of the subject. The invention also provides methods of inducing cell growth inhibition in a cancer cell line comprising inducing expression of Wwox in the cell line. In some embodiments, the cancer cell or cancer cell line is lung cancer.

The invention also provides polynucleotides comprising: a polynucleotide encoding a functional WWOX gene product; and a heterologous promoter operatively linked to the polynucleotide encoding the functional WWOX gene product. In some embodiments, the two ends of the polynucleotide are linked, resulting in a circular-polynucleotide.

The invention also provides vectors comprising a WWOX gene product expression cassette comprising: a polynucleotide encoding a functional WWOX gene product; and a heterologous promoter operatively linked to the polynucleotide encoding the functional WWOX gene product. In some embodiments, the vector is a viral vector, and in some embodiments, the viral vector is a recombinant adenoviral vector. The invention also provides cells comprising the viral vector according to the invention. The cells may be lung cells, and in particular, lung cancer cells.

The invention also provides pharmaceutical compositions for treating cancer in a subject, comprising: a viral vector, said vector comprising a WWOX gene product expression cassette, said cassette comprising a polynucleotide encoding a functional WWOX gene product and a heterologous promoter operatively linked to the polynucleotide encoding said functional WWOX gene product; and a pharmaceutically acceptable excipient. The viral vector may be, for example, a recombinant adenoviral vector. In some embodiments, the composition is formulated for inhalation.

The invention still further provides a plasmid, comprising: a polynucleotide encoding a functional WWOX gene product; and a heterologous promoter operatively linked to the polynucleotide encoding said functional WWOX gene product. The invention also provides cells comprising the plasmid according to the invention.

The invention also includes methods of treating cancer in a subject, comprising administering to the subject a therapeutic compound capable of reactivating a WWOX gene. In some embodiments, the subject is a human. In some embodiments, the reactivation of the WWOX gene results in induction of apoptosis.

Additional features and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplar), and explanatory only and are not restrictive of the invention, as claimed.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1. Expression of Wwox protein. (A) Expression of endogenous Wwox is detected in U2020 and MCF7 cells but not in H1299, H460, or A549 cells (50 μg of proteins loaded). Lane 1, H1299; lane 2, H460; lane 3, A549; lane 4, U2020; lane 5, 14CF-7. (B) Expression of Wwox after infection with Ad-WWOX (25 μg loaded). Lane 1, H1299, Ad-WWOX-infected; lane 2, H1299, Ad-GFP-infected; lane 3, H1299; lane 4, H460, Ad-WWOX-infected; lane 5, H460, Ad-GFP-infected; lane 6, H460; lane 7, A549, Ad-WWOX-infected; lane 8, A549, Ad-GFP-infected; lane 9, A549.

FIG. 2. Flow cytometry, analysis of untreated, Ad-GFP-, and Ad-WWOX-infected cells. Wwox-negative A549, H460, and H1299 cells undergo apoptosis 5 days after restoration of Wwox expression by Ad-WWOX infection, but U2020 cells are unaffected. Ad-GFP infection did not induce apoptosis.

FIG. 3. Effect of Wwox expression on cell growth in vitro. (A) Growth of uninfected, Wwox-negative A549, H460, and H1299 cells, and cells after infection with Ad-GFP and Ad-WWOX. (B) Immunoblot detection of PARP and caspase 3. Lane 1, A549; lane 2, A549/Ad-GFP; lane 3, A549/Ad-WWOX; lane 4, H460; lane 5, H460/Ad-GFP; lane 6, H460/Ad-Wwox; lane 7, H1299; lane 8, H1299/Ad-GFP; lane 9, H1299/Ad-WWOX; lane 10, U2020; lane 11, U2020/Ad-GFP; lane 12, U2020/Ad-WWOX. PARP is cleaved in Wwox-negative cell lines when Wwox is restored through Ad-Wwox infection (lanes 3, 6, and 9). Caspase 3 is cleaved in A549 and H460 (lanes 3 and 6) but not in H1299 cells after Ad-WWOX infection. In U2020 cells, neither PARP nor caspase 3 is cleaved after Ad-WWOX infection (lane 12).

FIG. 4. Inducible expression of Wwox in H1299/I cells. (A) Cells were cultured in the presence (+) or absence (−) of 10 μM ponA for 48 hr and tested for Wwox expression. Clones 7 and 2, which expressed the transgene only upon induction with ponA, were used in subsequent experiments. GAPDH expression served as loading control. (B) H1299/I clone 7 cells incubated in the absence or presence of increasing concentrations of ponA for 48 hr. Wwox levels increased in a dose-dependent manner and were quantified by densitometry, normalized to GAPDH expression levels. (C) Time course of Wwox induction in H1299/I clone 7 cells after treatment with 10 μM ponA. Wwox levels were quantified by densitometry. (D) Effect of 10 μM ponA on growth of H1299/I clone 7 cells. On day 1, ponA was added, and maximum Wwox expression was found on day 4. From day 5, the induced cells (H1299/I⁺) grow significantly more slowly than uninduced cells (H1299/I⁻)(P<0.001). The experiment was done in triplicate.

FIG. 5. Effect of Wwox expression on tumorigenicity of lung cancer cells. (A) Tumor volume of untreated, Ad-GFP-, and Ad-WWOX-infected A549, H460, and U2020 lung cancer cells. Restoration of Wwox expression in A549 and H460 cells suppressed tumor growth significantly (P<0.001) compared with Ad-GFP infected cells. (B) Tumor volume of untreated, Ad-GFP-, and Ad-WWOX-infected H1299 cells and H1299/I⁻ and H1299/I⁺ cells. Tumors were suppressed in Ad-WWOX-infected H1299 cells and in H1299/I⁺ cells. (C) Examples of tumor formation by uninfected, Ad-GFP-, and Ad-WWOX-infected A549, H1299/I⁻, and H1299/I⁺ cells.

FIG. 6. Ex vivo analysis of H1299/I⁻ and H1299/I⁺ cells. (A) Protein lysates from H1299 (lane 1), uninduced H1299/I⁻ (lanes 2, 3, and 4), and induced H1299/I⁺ (lane 5) tumors tested for Wwox expression by immunoblot analysis. Wwox was not expressed in the H1299/I⁻ or H1299/I⁺ tumors. (B) A portion of the H1299I/⁺ tumor was plated and cultured, and cells were treated with ponA. Wwox was reexpressed after 48 hr of treatment with 10 μM ponA, indicating the presence of the inducible WWOX plasmid.

FIG. 7 Table 1—Tumor weight (in grams) ±SD in nude mice.

### DETAILED DESCRIPTION OF THE PREFERRED EMBODIMENT

Cell Culture. Wwox-negative A549, H460, and H1299 and Wwox-positive U2020 lung cancer cell lines from American Type Culture Collection were maintained in RPMI medium 1640 with 10% FBS. HEK-293 CymR cells from Qbiogene (Carlsbad, Calif.) were cultured in DMEM with 10% FBS. H1299 cells do not express p53, whereas A549 and H460 express wild-type p53 (20).

Recombinant Ads and in Vitro Transduction. WWOX cDNA from normal human liver RNA (Ambion, Austin, Tex.) was reverse-transcribed by SuperScript First-Strand Synthesis (Invitrogen). Double-stranded cDNA was prepared by PCR amplification using the following conditions: 95° C. for 3 min. 30 cycles at 94° C. for 30 sec. 65° C. for 60 sec. 72° C. for 30 sec, and 72° C. for 7 min; WWOX forward 5′-GCCAGGTGCCTCCACAGTCAGCC-3′ and WWOX reverse 5′-TGTGTGTGCCCATCCGCTCTGAGCTCCAC-3′ primers were used. The cDNA was cloned into Adenovator-CM5(CuO)-IRES-GFP transfer vector (Qbiogene) (11). This vector allows transgene expression driven by the cumate-inducible CMV5(CuO) promoter. An internal ribosome entry site sequence ensures coexpression of GFP. The recombinant plasmid, Ad-WWOX, was transfected into modified human fetal kidneys HEK-293 CymR cells (Qbiogene) constitutively expressing the CymR protein, which represses the CMV5(CuO) promoter and expression of Wwox during packaging and expansion of the WWOX Ad. After 14-21 days, homologous recombination occurred in cells, leading to plaque formation. Plaques were isolated, and viruses were amplified in HEK-293 CymR cells and purified by CsCl gradient centrifugation. Titers were determined by absorbance measurement (number of viral particles per ml) and plaque assay (plaque-forming units/ml), and trans gene expression was assessed by immunoblot using Wwox monoclonal antibody (21). Cells were transduced with recombinant Ads at increasing multiplicities of infection (mois) (number of viral particles per cell), and transduction efficiency was determined by visualization of GFP-expressing cells.

Inducible WWOX Transfectants. The human WWOX cDNA was cloned into BamHI and EcoRI sites of the pIND vector. H1299 cells were transfected with 10 μg of pVgRXR vector, which contains the ecdysone nuclear receptor subunits, and clones were selected and tested for ponasterone A (ponA)-inducible expression by transient transfection with a reporter plasmid. Clones showing the highest expression were transfected with 10 μg of the pIND-WWOX vector and cultured in zeocin (150 μg/ml) and G418 (1,200 μg/ml). H1299/I clones were selected and tested for inducible WWOX expression after ponA (5-10 μM) treatment.

Western Blot Analysis. Protein extraction and immunoblot analysis were performed as described in ref. 13. The following primary antisera were used: mouse monoclonal anti-Wwox, 1:500; rabbit polyclonal anti-caspase 3, 1:1,000 (Cell Signaling Technology, Beverly, Mass.); rabbit polyclonal anti-caspase 9, 1:200 (Santa Cruz Biotechnology); mouse monoclonal anti-caspase 8 (Cell Signaling Technology), 1:1,000; rabbit polyclonal anti-PARP [poly(ADP-ribose) polymerase], 1:1,000 (Cell Signaling Technology); and rabbit polyclonal anti-β-actin, 1:1,000 (Cell Signaling Technology).

Cell Growth and Cell Cycle Kinetics. Cells (2×10⁵) were infected at mois of 10, 25, 50, 75, and 100 and, at 24 hr intervals, were harvested, stained with trypan blue, and counted (ViCell counter, Beckman Coulter). For flow cytometry, cells were harvested 5 days after infection, fixed in cold methanol, RNase-treated, and stained with propidium iodide (50 μg/ml). Cells were analyzed for DNA content by EPICS-XL scan (Beckman Coulter) by using doublet discrimination gating. All analyses were performed in duplicate.

In Vivo Studies. Animal studies were performed according to institutional guidelines. H460, A549, and U2020 cells were infected in vitro with Ad-WWOX (moi=100) or Ad-GFP or were mock-infected. At 24 hr after infection, 5×10⁶ viable cells were injected s.c. into left flanks of 6-week-old female nude mice (Charles River Breeding Laboratories), five mice per infected or control cell line. H1299 cells were infected in vitro with Ad-GFP or Ad-WWOX at a moi of 100. H1299/I cells were treated with 10 μM ponA (H1299/I⁺ cells) to induce Wwox expression. Tumorigenic controls were uninduced cells (H1299/I⁻). Induced (H1299/I, 24 hr after ponA treatment) and uninduced (10⁷) cells were injected into five nude mice; five mice were also injected with Ad-WWOX, Ad-GFP, and mock-infected H1299 cells. Tumor diameters were measured every 5 days, and tumors were weighed after necropsy. Tumor volumes were calculated by using the equation V (in mm³)=a×b²/2, where a is the largest diameter and b is the perpendicular diameter.

Ex Vivo Studies. Protein lysates from tumors of H1299, H1299/I⁻, and H1299/I⁺ injected mice were evaluated for Wwox expression by immunoblot analysis. Fragments from H1299/I⁺ tumors were cultured and treated with 10 μM ponA for 2 days to detect expression of inducible Wwox by immunoblot.

Statistical Analysis. Results of in vitro and in vivo experiments were expressed as mean±SD. Student's two-sided t test was used to compare values of test and control samples. P<0.05 indicated significant difference.

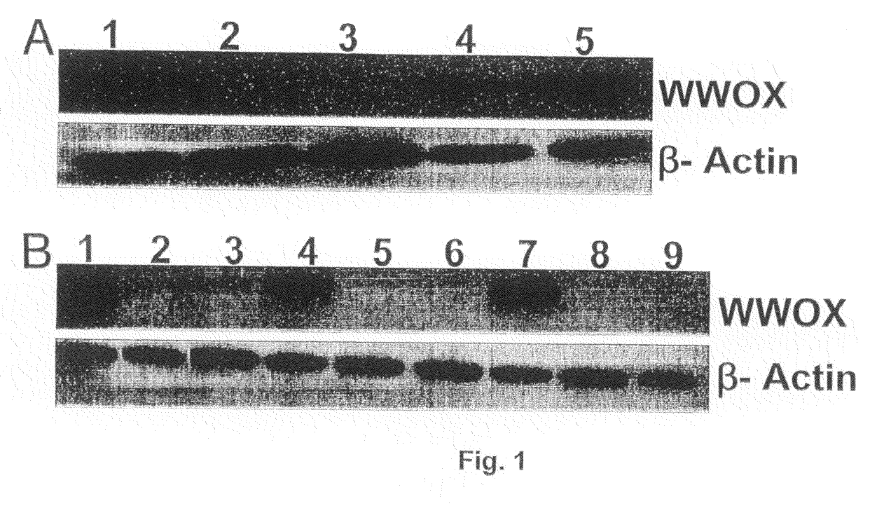

Wwox Expression in Parental and Ad-WWOX-Infected Lung Cancer Cells. Immunoblot analysis of proteins of lung cancer cell lines showed that A549, H460, and H1299 cells did not express endogenous Wwox, whereas Wwox was detected in U2020 cells. Breast cancer MCF-7 cells express abundant endogenous Wwox (18) and served as a positive control (FIG. 1A).

Lung cancer cells were infected with Ad-WWOX or Ad-GFP at a moi of 100; the adenoviral transgene was expressed in nearly 100% of cells of each cell line, as assessed by confocal microscopy of GFP fluorescence (data not shown). Immunoblot analysis 72 hr after infection showed Wwox overexpression in all Ad-WWOX-transduced cells (FIG. 1B).

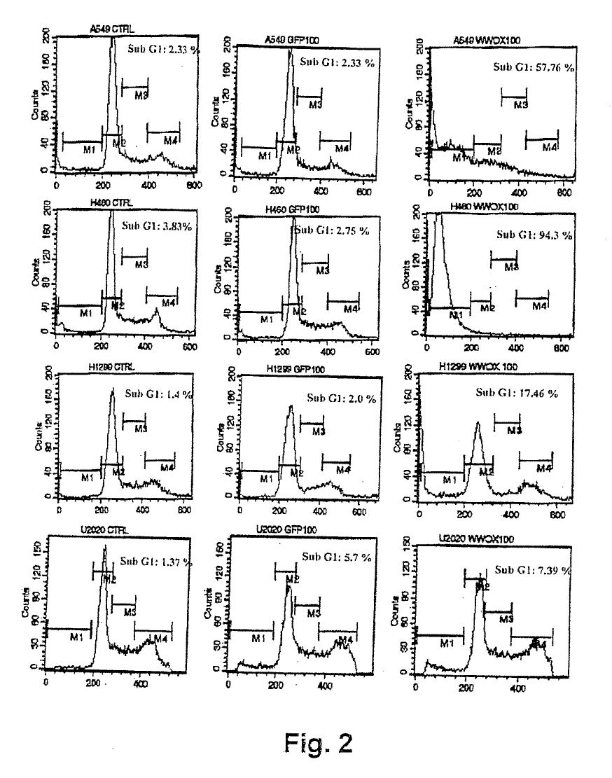

Cell Cycle Kinetics of Infected Cells. Cell cycle alterations induced by Wwox overexpression were assessed after infection at several mois, with Ad-WWOX or Ad-GFP. A sub-G₁ population was observed after Ad-WWOX infection in A549, H460, and H1299 cells that do not express endogenous Wwox but not in endogenous Wwox-positive U2020 cells. Ad-GFP infection did not modify cell cycle profiles. At 96 hr after Ad-WWOX infection (moi=100), 58% of A549, 94% of H460, and 17% of H1299 cells were in the sub-G₁ fraction; 7% of U2020 cells were in the sub-G₁ fraction (FIG. 2). Wwox induction of cell death was moi- and time-dependent (data not shown).

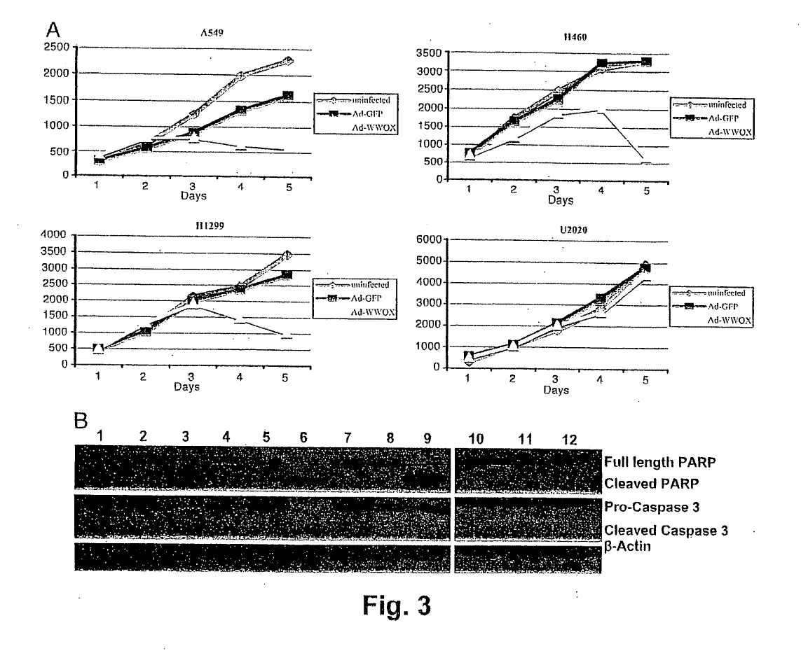

Apoptotic Pathways in Wwox-Reexpressing Cells. A549, H460, H1299, and U2020 lung cancer cell lines were infected with increasing mois, and the fraction of transduced cells was monitored by confocal microscopy and cell cycle kinetics analyses. Significant differences were observed in cell growth for Ad-WWOX and Ad-GFP infection, at a range of mois, in lung cancer cell lines (A549, H460, and H1299) lacking endogenous Wwox (FIG. 3A). U2020 cells were unaffected by exogenous Wwox expression.

To study Wwox-induced apoptotic pathways, expression of downstream apoptotic effectors was assessed in vitro. At 96 hr after infection, pro-caspase 3 and full-length PARP-1 levels were reduced in Ad-WWOX-infected A549 and H460 cells compared with Ad-GFP control cells. In H1299 cells, a decrease of full-length PARP-1 was observed. Cleavage of precursors was not observed in infected U2020 cells (FIG. 3B).

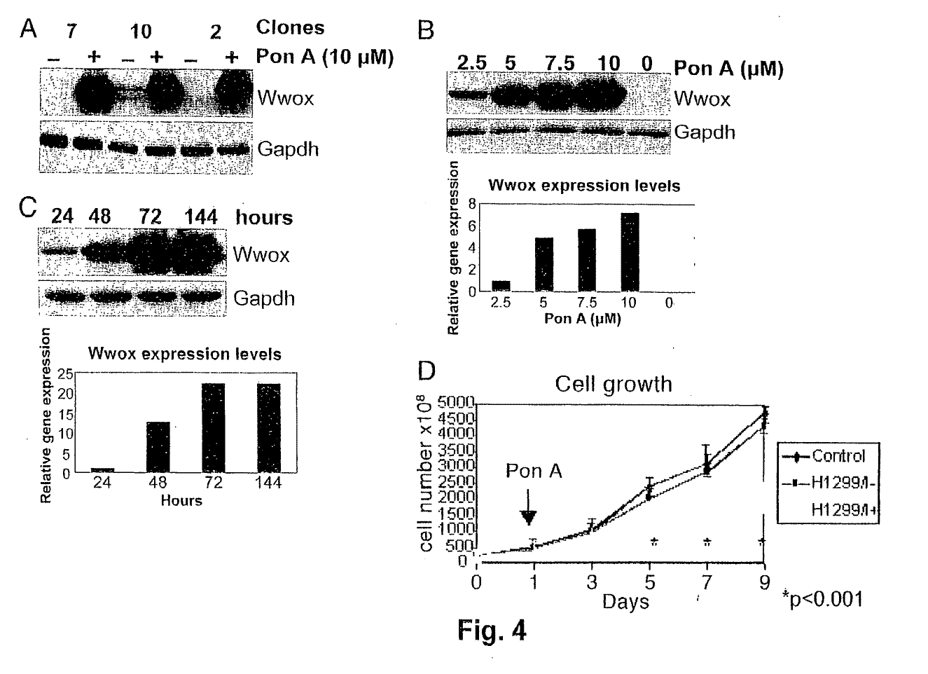

Effects of Conditional Wwox Expression in H1299 Cells. H1299/I clone 7 expressed the WWOX transgene only on induction with ponA (FIG. 4A) and was used in subsequent experiments. Wwox expression increased in a dose-dependent manner after ponA treatment (FIG. 4B) from 24 to 72 hr (FIG. 4C).

Clone 7H1299/I⁻ (uninduced) cells were plated, and, 24 hr later (day 1) Wwox expression was induced by 10 μM ponA. Maximum expression was observed at day 4 and significantly affected cell proliferation by day 5 (FIG. 4D), causing reduction in cell numbers and suggesting that Wwox inhibits growth of H1299 cells.

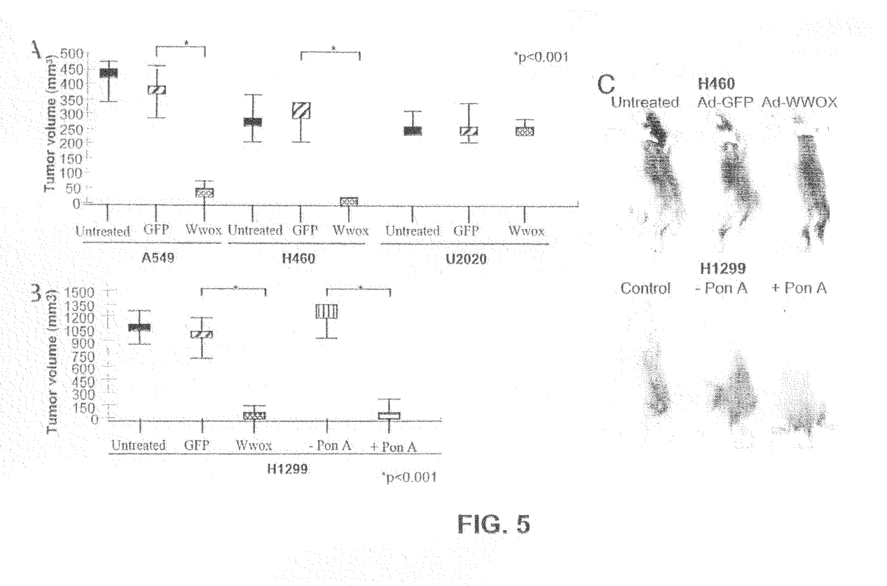

Tumorigenicity of Ad-WWOX-Infected Lung Cancer Cell Lines. Nude mice were inoculated with 5×10⁶ A549, H460, and U2020 cells infected in vitro at a moi of 100 with Ad-GFP or Ad-WWOX and cultured for 24 hr. Uninfected cells served as tumorigenic controls. At 28 days after injection, tumor growth was completely suppressed in mice inoculated with Ad-WWOX-infected H460 cells (FIG. 5A). The average tumor weights for controls (Ad-GFP and untreated H460 cells) at day 28 were 0.61±0.15 g and 0.64±0.11 g, respectively. At 28 days, two of five mice inoculated with Ad-WWOX-infected A549 cells showed no tumors, and average tumor weight was 0.08-0.03 g, significantly lower (P<0.001) than tumors of Ad-GFP-infected A549 (0.81±0.16 g) and mock-infected A549 (0.86±0.15 g) cells (Table 1). In mice injected with infected U2020 cells, no tumor growth suppression was observed (FIG. 5A).

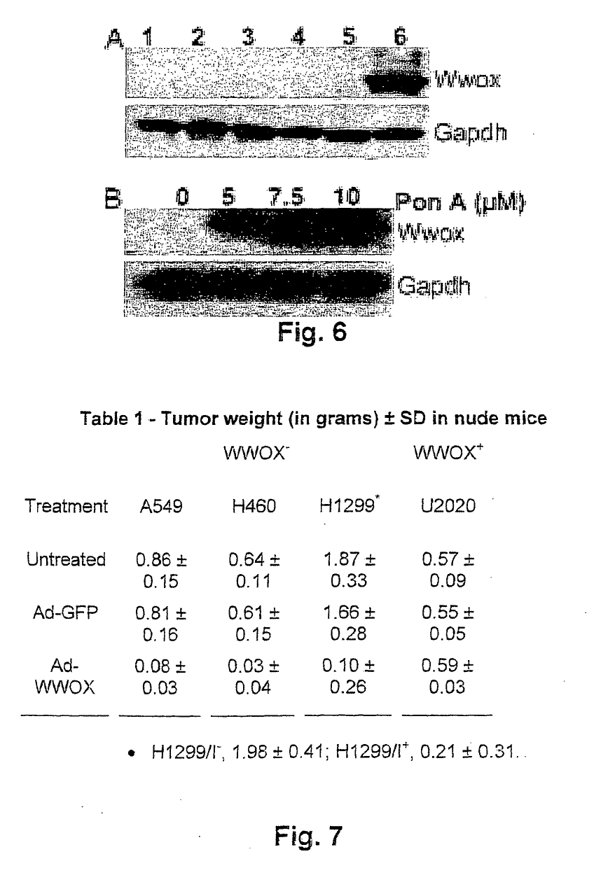

Effect of Induced Wwox Expression on Tumorigenicity. We next compared tumorigenicity, of H1299 cells infected with Ad-WWOX or induced to express Wwox by ponA treatment. Nude mice were inoculated with 1×10⁷ cells 24 hr after infection with Ad-WWOX or Ad-GFP. Five mice were also injected with 1×10⁷ uninduced H1299/I (H1299/I⁻) and 10⁷ H1299/I⁺ cells 24 hr after ponA treatment. At 28 days after injection, three of five and four of five mice inoculated with Ad-WWOX-infected H1299 cells and H1299/I⁺ cells, respectively, displayed no tumors (FIG. 5B). Average weight of tumors from Ad-WWOX-infected (0.10±0.26 g) and H1299/I⁺ (0.21±0.31 g) cells was significantly reduced compared with tumors from Ad-GFP (1.66±0.28 g), H1299/I⁻ (1.98±0.41 g), and parental H1299 (1.87±1.33 g) cells (FIG. 7—Table, 1). Thus, Wwox expression, delivered by viral infection (Ad-WWOX) or by induction of expression of an inactive “endogenous” WWOX gene (H1299/I⁺), was effective in suppressing lung cancer cell growth in nude mice.

Wwox Expression in H1299/I⁺ Explanted Tumors. To assess Wwox expression ex vivo, we performed immunoblot analysis of proteins extracted from fragments originating from parental H1299, H1299/I⁻, and H1299/I⁺ tumors; Wwox expression was not found in any of the tumors (FIG. 6A). Explanted, cultured fragments from H1299/I⁺ tumors were examined for retention of inducible WWOX plasmid by treating with ponA and testing for Wwox expression by immunoblot analysis. The detection of Wwox induction in H1299/I⁺ explants revealed that the WWOX plasmid was present and inducible (FIG. 6B), suggesting that the small tumors were derived from inoculated cells that had lost expression of Wwox due to absence of inducer in vivo.

Discussion

Innovative therapeutic strategies are urgently needed for lung cancer treatment. Because genes at common fragile sites are frequently inactivated early in the neoplastic process, especially on exposure to environmental carcinogens, we have been interested in the effect of loss of fragile gene expression in development of cancer and therapeutic effects of their restoration (22). A number of studies have suggested that the fragile WWOX gene is inactivated in a significant fraction of lung cancers (10, 16), particularly by promoter hypermethylation (16). Hypermethylation is reversible, a strategy with promise for cancer therapy. Thus, we have determined whether restoration of Wwox expression in lung cancer cells lacking expression of endogenous Wwox would reverse malignancy despite numerous cancer-associated genetic alterations that have accumulated in lung cancer cell lines. We haste restored Wwox expression in four lung cancer cell lines by infection with Ad-WWOX and observed dramatic loss of tumorigenicity of the lung cancer cells that lacked endogenous Wwox.

Introduction of the WWOX gene in the three Wwox-negative cell lines resulted in induction of apoptosis in vitro, as shown by the fraction of cells with sub-G₁ DNA content and by suppression of cell growth in culture. The fraction of Ad-WWOX-infected H1299 cells with sub-G₁ DNA content was lower than for the other two WWOX-negative cell lines, possibly because apoptosis may occur later after restoration of Wwox expression in H1299 cells; another possibility is that expression of p53 in A549 and H460 cells had an additive effect with expression of Wwox protein, although the tumor suppressive effect was similar in the three lung cancer cell lines. The U2020 lung cancer cells expressing endogenous Wwox were not affected by overexpression of Wwox, suggesting that normal Wwox-expressing lung cells would be unaffected bit Wwox overexpression after WWOX gene therapy. Growth of all three lung cancer cells in vitro was adversely affected by overexpression of Wwox after virus infection or ponA induction, as shown by the downturn in cell number after a few days of Wwox overexpression. It will be interesting to examine Wwox binding to know interacting proteins at days 2-5 in these in vitro overexpression cultures to define the signal events directly downstream of Wwox expression after WWOX infection or induction.

We observed efficient suppression of in vivo tumorigenicity of lung cancer cell lines by Ad-WWOX transduction in three WWOX-negative lung cancer cell lines and by induction of Wwox expression in stably transfected H1299 lung cancer cells. The tumorigenicity of the aggressive H460 cell line was completely suppressed by Ad-WWOX treatment at 28 days after injection. A significant reduction in tumor occurrence and size was observed in animals injected with WWOX-transduced A549 and H1299 cells. The results suggest that Wwox loss may play an important role in the pathogenesis of lung cancer. It is interesting that both methods of Wwox restoration in H1299 cells appeared to result in more dramatic effects in vivo than in vitro, possibly because the in vivo microenvironment somehow activates the Wwox apoptotic pathway.

This study demonstrates that WWOX induces cell growth inhibition and apoptosis in lung cancer cells. In A549 and H460 cell lines, we observed caspase-dependent induction of apoptosis through the intrinsic pathway. In H1299 cells, we observed cleavage of full-length PARP-1, but procaspase 3, 9, and 8 were not cleaved, possibly because apoptosis occurs later in these cells. Wwox and Fhit protein expression is frequently reduced in lung, breast, and bladder cancers in association with promoter hypermethylation (16). Epigenetic alterations can be reversed by specific agents or inhibitors, suggesting such inhibitors as therapeutic agents (23-26). The ponA-inducible expression of Wwox can be considered a model for the effects of WWOX reactivation after silencing by epigenetic mechanisms. The extent of loss of tumorigenicity after restoring inducible Wwox expression was comparable to the tumor suppression observed after Ad-WWOX expression, both in vitro and in vivo, suggesting that massive overexpression of Wwox is not necessary to effect tumor suppression. This finding suggests that drugs capable of reactivating the epigenetically silenced WWOX gene could be effective in treatment of lung cancer.

The restoration of Wwox protein expression in lung cancer cells is followed by induction of apoptosis in vitro and suppression of tumorigenicity in vivo and suggests that reactivation of the Wwox signal pathway is a potential target for lung cancer prevention and therapy.

In accordance with the provisions of the patent statutes, the principle and mode of operation of this invention have been explained and illustrated in its preferred embodiment. However, it must be understood that this invention may be practiced otherwise than as specifically explained and illustrated without departing from its spirit or scope.

### REFERENCES

The references discussed above and the following references, to the extent that they provide exemplary procedural or other details supplementary to those set forth herein, are specifically incorporated herein by reference.
1. Greenlee, R. T., Hill-Harmon, M. B., Murray, T. & Thun, M. (2001) CA Cancer J. Clin. 51, 15-36.
2. Roth, J. A., Nguyen, D. Lawrence, D. D., Kemp, B. L. Carrasco, C. H., Ferson, D. Z., Hong, W. K., Komaki, R., Lee, J. J., Nesbitt, J. C., et al. (1996) Nat. Med. 2, 985-991.
3. Nemunaitis, J., Swisher, S. G., Timmons, T., Connors, D. Mack, M., Doerksen, L., Weill, D., Wait, J., Lawrence, D. D., Kemp, B. L., et al. (2000) J. Clin. Oncol. 18, 609-622.
4. Roth, J. A., Swisher, S. G., Merritt, J. A., Lawrence, D. D., Kemp, B. L., Carrasco, C. H., El-Naggar, A. K., Fossella, F. V., Glisson, B. S., Hong, W. K., et al. (1998) Semin. Oncol. 25, Suppl. 8, 33-37.
5. Weill, D., Mack, M., Roth, J., Swisher, S., Proksch, S., Merritt, J. & Nemunaitis, J. (2000) Chest 118, 966-970.
6. Swisher, S. G., Roth, J. A., Nemunaitis, J., Lawrence, D. D., Kemp, B. L., Carrasco, C. H., Connors, D. G., El-Naggar, A. K., Fossella, F., Glisson, B. S., et al. (1999) J. Natl. Cancer Inst. 91, 763-771.
7. Griscelli, F., Opolon, P., Saulnier, P., Mami-Chouaib, F., Gautier, E., Echchakir, H., Angevin, E., Le Chevalier, T., Bataille, V., Squiban, P., et al. (2003) Gene Ther. 10, 386-395.
S. Sozzi, G., Pastorino, U. Moiraghi, L., Tagliabue, E., Pezzella, F., Girelli. C. Tomeili, S., Sard, L., Huebner, K., Pienotti, M. A., et al. (1998) Cancer Res. 58, 5032-5037.
9. Olita, M., Inouhe, H., Cotticeli, M. G., Kastur-, K., Baffa, R., Palazzo, J., Siprashvili, Z., Mori, M., McCue, P., Druck, T., et al. (1996) Cell 84, 587-597.
10. Yendamuri, S., Kuroli, T., Trapasso, F., Henry, A. C., Dumon, K. R., Huebner, K., Williams, N. N., Kaiser, L. R. & Croce, C. M. (2003) Cancer Res. 63, 878-881.
11. Bednarek, A. K., Laflin. K. J., Daniel, R. L., Liao, Q., Hawkins, K. A. & Aldaz. C. M. (2000) Cancer Res. 60, 2140-2145.
12. Aqeilan, R. I., Pekarslky, Y., Herrero, J. J., Palamarchuk, A., Letofskh, J. Druck, T., Trapasso, F., Han, S. Y., Metino, G., Huebner, K. & Croce, C. M. (2004) Proc. Natl. Acad. Sci. USA 101, 4401-4406.
13. Aqeilan, R. I., Palamarchulk, A., Weigel, R. J., Herrero, J. J. Pekarsky, Y. & Croce, C. M. (2004) Cancer Res. 64, 8256-8261.
14. Aqeilan, R. I., Donati, V., Palamarchuk, A., Trapasso, F., Pekarsky, Y., Sudol, M. & Croce, C. M. (2005) Cancer Res. 65, 6764-6772.
15. Driouch, K., Prydz, H., Monete, R., Johansen, H., Lidereau, R. & Frengen, E. (2002) Oncogene 21, 1832-1840.
16. Kuroki, T., Trapasso. F., Shiraishi, T., Alder, H., Mimori, K., Mori, M. & Croce, C. M. (2002) Cancee Res. 62, 2258-2260.
17. Paige, A., Taylor, K. J., Taylor, C., Hillier, S. G., Farrington, S., Scott, D., Porteous, D. J., Smyth, J. F., Gabra, H. & Watson, J. E. (2001) Proc. Natl. Acad. Sci. USA 98, 11417-11422.
18. Kuroki, T., Tandamuri, S., Trapasso, F. Matsuyama, A., Aqeilan, R. I., Alder, H., Rattan, S., Cesari, R., Nolli, M. L., Williams, N. N., et al. (2004) Clin. Cancer Res. 10, 2459-2465.
19. Iliopoulos, D. Guler, G., Han, S. Y., Johnston, D., Druck. T., McCorkell, K. A., Palazzo, J., McCue, P. A., Baffa, R. & Huebner, K. (2005) Oncogene 24, 1625-1633.
20. Nishizalci, M., Sasalki, J., Fang, B., Atkinson, E. N., Minna, J. D. Roth, J. A. & Ji, L. (2004) Cancer Res. 64, 5745-5752.
21. Milner, A. E. Levens, J. M. & Gregory, C. D. (1998) Methods Mol. Biol. 80, 347-354.
22. Roz, L., Gramegna, M., Ishii, H., Croce, C. M. & Sozzi. G. (2002) Proc. Natl. Acad. Sci. USA 99, 3615-3620.
23. Ingrosso, D., Cimmino, A., Pema, A. F., Masella, L., De Santo, N. G., De Bonis, M. L., Vacca, M., D'Esposito, M., D'Urso, M., Galletti, P. & Zappia, V. (2003) Lancet 361, 1693-1699.
24. McGregor, F., Muntoni, A., Fleming, J., Brown, J., Feli, D. H., MacDonald, D. G., Parkinson, E. K. & Harrison, P. R. (2002) Cancer Res. 16, 4757-4766.
25. Hennessy, B. T., Garcia-Manero, G., Kantarjian, H. M. & Giles, F. J. (2003) Expert Opin. Ivestig. Dregs 12, 1985-1993.
26. Takai, N., Desmond, J. C., Kumagai, T., Gui, D. Said, J. W., Whittaker, S., Miyakawa. I. & Koeffler, H. P. (2004) Clin. Cancer Res. 10, 1141-1149.

What is claimed is:

1. A method for treating cancer in a subject, comprising administering to the subject a polynucleotide encoding a functional WWOX gene product.

2. The method according to claim 1, wherein the cancer is chosen from lung cancer, breast cancer, ovarian cancer, prostate cancer, bladder cancer, esophageal cancer, and pancreatic cancer.

3. The method according to claim 2, wherein the cancer is lung cancer.

4. The method according to claim 1, wherein the subject is a human.

5. The method according to claim 1, wherein the administration comprises gene therapy.

6. The method according to claim 5, wherein the gene therapy comprises recombinant viral gene therapy.

7. The method according to claim 6, wherein the recombinant viral gene therapy comprises recombinant adenoviral gene therapy.

8. A method of treating cancer in a subject comprising inducing Wwox expression in at least one cancer cell of the subject.

9. A method of inducing cell growth inhibition in a cancer cell line comprising inducing expression of Wwox in the cell line.

10. The method according to claim 9, wherein the cancer cell line is lung cancer.

11. A polynucleotide comprising: a polynucleotide encoding a functional WWOX gene product; and a heterologous promoter operatively linked to the polynucleotide encoding the functional WWOX gene product.

12. The polynucleotide according to claim 11, wherein the two ends of the polynucleotide are linked, resulting in a circular polynucleotide.

13. A vector comprising a WWOX gene product expression cassette comprising:
a polynucleotide encoding a functional WWOX gene product; and
a heterologous promoter operatively linked to the polynucleotide encoding the functional WWOX gene product.

14. The vector according to claim 13, wherein the vector is a viral vector.

15. The vector according to claim 14, wherein the viral vector is a recombinant adenoviral vector.

16. A cell comprising the viral vector according to claim 14.

17. The cell according to claim 16, wherein the cell is a lung cell.

18. The cell according to claim 17, wherein the lung cell is a lung cancer cell.

19. A pharmaceutical composition for treating cancer in a subject, comprising:
a viral vector, said vector comprising a WWOX gene product expression cassette, said cassette comprising a polynucleotide encoding a functional WWOX gene product and a heterologous promoter operatively linked to the polynucleotide encoding said functional WWOX gene product; and
a pharmaceutically acceptable excipient.

20. The pharmaceutical composition according to claim 19, wherein the viral vector is a recombinant adenoviral vector.

21. The pharmaceutical composition according to claim 19, wherein the composition is formulated for inhalation.

22. A plasmid, comprising:
a polynucleotide encoding a functional WWOX gene product; and
a heterologous promoter operatively linked to the polynucleotide encoding said functional WWOX gene product.

23. A cell comprising the plasmid according to claim 22.

24. A method of treating cancer in a subject, comprising administering to the subject a therapeutic compound capable of reactivating a WWOX gene.

25. The method according to claim 24, wherein the subject is a human.

26. The method according to claim 24, wherein the reactivation of the WWOX gene results in induction of apoptosis.

27. A method of cancer therapy comprising restoration of Wwox expression in lung cancer cells lacking expression of endogenous Wwox, thereby reversing malignancy.

28. A method for inducing WWOX cell growth inhibition and apoptosis in lung cancer cells.
